(19) United States
(12) Patent Application Publication
(10) Pub. No.: US 2006/0167069 A1
(43) Pub. Date: Jul. 27, 2006

(54) Pharmaceutical composition of metaxalone with enhanced oral bioavailability

(75) Inventors: Nitin Bhalachandra Dharmadhikari, Mumbai (IN); Ashish Prabhakar Mungre, Mumbai (IN); Yashoraj Rupsinh Zala, Mumbai (IN)

Correspondence Address: BANNER & WITCOFF, LTD., TEN SOUTH WACKER DRIVE, SUITE 3000, CHICAGO, IL, 60606, US

(73) Assignee: Sun Pharmaceutical Industries Ltd., Mumbai, Maharashtra (IN)

(21) Appl. No.: 10/526,285

(22) PCT Filed: Sep. 2, 2003
(86) PCT No.: PCT/IN03/00294

§ 371 Date: Mar. 2, 2005

(30) Foreign Application Priority Data
Sep. 2, 2002 (IN) 790/MUM/2002

(51) Int. Cl. A61K 31/421

(52) U.S. Cl. 514/376

(57) ABSTRACT

The present invention provides a pharmaceutical composition comprising metaxalone and pharmaceutically acceptable excipients, characterized in that the pharmaceutical composition has enhanced oral bioavailability. The present invention also provides a pharmaceutical composition comprising metaxalone and pharmaceutically acceptable excipients, characterized in that the extent of absorption of metaxalone is independent of whether the composition is administered to the patient with food or on an empty stomach.

### FIELD OF THE INVENTION

The present invention relates to a pharmaceutical composition of metaxalone with enhanced oral bioavailability. The present invention further relates to a pharmaceutical composition of metaxalone from which the extent of absorption of metaxalone, more preferably, the bioavailability (the rate and extent of absorption), is independent of whether the composition is administered to the patient with food or on an empty stomach.

### BACKGROUND OF THE INVENTION

Metaxalone, [5-(3,5-dimethylphenoxymethyl)-2-oxazolidinone] disclosed in the U.S. Pat. No. 3,062,827 is indicated as an adjunct to rest, physical therapy, and other measures for the relief of discomforts associated with acute, painful musculo-skeletal conditions. Metaxalone is therapeutically categorized as a skeletal muscle relaxant. Its mechanism of action in humans has not been well established, but may be due to general central nervous system depression. Metaxalone does not directly relax tense muscles in man. The recommended dose of metaxalone for adults and children over 12 years of age is two tablets (800 mg) three to four times daily.

U.S. Pat. No. 3,062,827 discloses crystalline metaxalone with a melting point of 121.5-123° C. Metaxalone, being a hydrophobic molecule, has low aqueous solubility, which in turn affects its bioavailability.

U.S. Pat. No. 6,407,128 discloses methods of increasing the oral bioavailability of metaxalone by administration of an oral dosage form with food in human subjects. Preferably the invention describes administration of the dosage form between 30 minutes prior, to 2 hours after consuming solid food with sufficient bulk and fat content that is not rapidly dissolved and absorbed in the stomach. Thus, in the disclosed invention, a method of increasing rate and extent of metaxalone absorption is provided comprising administering the therapeutically effective amount of metaxalone in the formulation of the drug product Skelaxin® to the patients with food.

Thus, the prior art discloses that metaxalone has low aqueous solubility, has a high dose and its oral bioavailability is affected by the presence of food. These factors point towards a bioavailability which is limited by the ability of the pharmaceutical composition to release metaxalone at a rapid rate in an absorbable form. However, the prior art does not provide a pharmaceutical composition of metaxalone with enhanced or improved oral bioavailability. Enhanced oral bioavailability of drug substance is known to increase both onset of action and therapeutic efficacy of the drug. Hence, it is desirable to provide metaxalone in a pharmaceutical composition with enhanced bioavailability as compared to commercially available pharmaceutical compositions of metaxalone.

The prior art also does not provide any pharmaceutical composition of metaxalone from which the extent of absorption of metaxalone, more preferably, the bioavailability (the rate and extent of absorption), is independent of whether the composition is administered to the patient with food or on an empty stomach. The desired bioavailability is exceeded if the patient takes the composition with food exposing the patient to higher blood level and amounts of metaxalone.

### OBJECTS OF THE INVENTION

It is the object of the present invention to provide a pharmaceutical composition comprising metaxalone and pharmaceutically acceptable excipients, characterized in that the pharmaceutical composition has enhanced oral bioavailability.

Yet another object of the present invention is to provide a pharmaceutical composition comprising metaxalone and pharmaceutically acceptable excipients, from which the extent of absorption of metaxalone, more preferably, the bioavailability (the rate and extent of absorption), is independent of whether the composition is administered to the patient with food or on an empty stomach.

### BRIEF DESCRIPTION OF THE DRAWING

FIG. 1 shows the plasma concentration vs. time profile obtained upon administration of an embodiment of the pharmaceutical composition of the present invention having 400 mg metaxalone, in comparison to that obtained from an equivalent dose of a conventional pharmaceutical composition available commercially.

### DETAILED DESCRIPTION OF THE INVENTION

The present invention provides a pharmaceutical composition comprising metaxalone and pharmaceutically acceptable excipients, characterized in that the pharmaceutical composition has an enhanced bioavailability as compared to the conventional pharmaceutical compositions of metaxalone known in the art and commercially available.

In one embodiment of the present invention, the metaxalone that is used is a pharmaceutically acceptable solubility-improved form. The term “pharmaceutically acceptable solubility-improved form” as used herein includes micronised metaxalone, salt form of metaxalone, high-energy crystalline form of metaxalone or amorphous metaxalone. The solubility-improved form may be obtained by methods known in the art such as milling, crystallisation, sublimation, spray drying, and the like, of the drug substance alone, or by known methods of forming solid dispersion of the drug in a carrier, for example, melt dispersion, solvent evaporation, spray coating the drug-carrier mixture on units providing surface for deposition of the dispersion, such as pellets, beads, granules, tablets and the like. In another embodiment, the composition comprises a mixture of metaxalone either in its low solubility form or in solubility-improved form, and an excipient that improves the solubility of metaxalone. Preferably, the excipient is a solubilizing agent. The solubilizing agent may be selected from the group consisting of surfactants, pH control agents, glycerides, partial glycerides, glyceride derivatives, polyoxyethylene and polyoxypropylene ethers and their copolymers, sorbitan esters, polyoxyethylene sorbitan esters and alkyl sulfonates. The pH control agents that may be used in the present invention include buffers, organic acids, organic acid salts, organic and inorganic bases, and organic and inorganic base salts. Alternately, the excipient may be a complexing agent, such as cyclodextrin.

In one preferred embodiment, the solubility-improved form of metaxalone used is micronized metaxalone. Micronised metaxalone may be obtained either by crystallisation of metaxalone or spray drying or by the use of conventional milling techniques. Where milling is employed, metaxalone may be micronized to the desired particle size range by milling in mills known in the art, for example, ball mill, rod mill, hammer mill, cutter mill, fluid energy attrition mill, jet mill, chaser mill, centrifugal-impact mill, roller mill, colloidal mill, microfluidizer, homogenizers, ultrasonic means and the like. The milling may be dry or wet milling of metaxalone and may be carried out in the absence or in the presence of pharmaceutically acceptable excipients. Typically, the volume size distribution of metaxalone that may be used in the present invention is given in Table 1 below.

TABLE 1

| % Undersize | Preferred | | More Preferred | | Most Preferred | |
|---|---|---|---|---|---|---|
| 99% | NMT* 40 | μm | NMT 20 | μm | NMT 10 | μm |
| 90% | NMT 30 | μm | NMT 14 | μm | NMT 6 | μm |
| 50% | NMT 10 | μm | NMT 5 | μm | NMT 3 | μm |

*NMT = Not More Than

Particle size distribution of metaxalone and values determined thereupon, as referred to herein, are those derived from measurement using a Sympatec HELOS(H0899) particle size analyzer. However, these may be measured by any suitable technique.

Micronised metaxalone having a specific surface area per unit volume of more than about 1.5 m²/cm³, preferably more than about 2.5 m²/cm³ may be used in preferred embodiments of the present invention. In highly preferred embodiments, the specific surface area per unit volume of metaxalone is equal to or more than about 3.0 m²/cm³.

Metaxalone may be used in the pharmaceutical compositions of the present invention in the range of about 400 mg to about 1600 mg. In particular, the pharmaceutical compositions of the present invention may have 400 mg of metaxalone.

The pharmaceutical composition of the present invention may be formulated into any suitable dosage form, such as tablets, capsules, pills, lozenges, granules, powders, pellets, liquids, emulsion, suspension, elixir and the like. The pharmaceutically acceptable excipients may be any pharmaceutical excipient that would function as carrier materials, bulking agents, binders, lubricants, buffer, surfactant, diluent, disintegrant, glidant, colouring agent and the like.

Pharmaceutically acceptable excipients that may be used in the present invention may be selected from those referred to in “The Handbook of Pharmaceutical Excipients”, third edition, Ed. by Arthur H. Kibbe; American Pharmaceutical Association, Washington D.C. (2000), as well as in “Remington: The Science and Practice of Pharmacy”, edition 20, Lippincott Williams and Wilkins, Philadelphia (2000).

One embodiment of the present invention may be prepared by a process which comprises mixing metaxalone in a solubility-improved form and pharmaceutically acceptable excipients, for example, binders such as cellulose derivatives, starch, gelatin, sugars, polyvinyl pyrrolidone and the like; disintegrants such as starch, modified starch such as sodium carboxymethyl starch, cellulose derivatives, natural and synthetic gums and the like; lubricants such as talc, magnesium stearate, colloidal silicon dioxide, polyethylene glycol and mixtures thereof; wetting agents such as polyols, surfactants and the like; colouring agents including food grade dyes and food grade dyes adsorbed onto a suitable adsorbent, such as clay or aluminium oxide; and formulating into a suitable dosage form by conventional means well known to a person skilled in the art.

In another embodiment of the present invention, the metaxalone and a pharmaceutically acceptable excipient may be together subjected to milling by conventional techniques known in the art like for example, using a ball mill, rod mill, hammer mill, cutter mill, fluid energy attrition mill, jet mill, chaser mill, centrifugal-impact mill, roller mill, colloidal mill, microfluidizer, homogenizers, ultrasonic means and the like and formulated into a suitable dosage form by conventional means well known to the person skilled in the art.

When the pharmaceutical composition of the present invention is formulated into tablets, methods well known to those skilled in the art are used such that the tablets readily disintegrate into granules which then readily disintegrate into easily wettable microparticles in order to effectively expose the surface of metaxalone to the surrounding gastro-intestinal fluids or alternatively the tablets rapidly erode exposing easily wettable microparticles. Factors affecting such performance of tablets are well known to those skilled in the art and include for example hardness of tablets, amounts and type of binding, disintegrating and lubricating agents used, use of wetting agents, moisture content of the granules etc. Preferably the composition of the present invention includes a wetting agent to improve the wettability of metaxalone. Preferably the wetting agent is an orally pharmaceutically acceptable excipient such as a polyol like polyethylene glycol and the like and surfactants such as nonionic, ionic surfactants, polyoxyethylene sorbitan esters, polyoxyethylene alkyl ethers, polyoxyethylene stearates, poloxamers and the like, and any other wetting agent known in the art. More preferably the surfactant is sodium lauryl sulfate.

The present invention provides a pharmaceutical composition comprising metaxalone and pharmaceutically acceptable excipients, characterized in that the extent of absorption of metaxalone is independent of whether the composition is administered to the patient with food or on an empty stomach. The present invention also provides a pharmaceutical composition comprising metaxalone and pharmaceutically acceptable excipients, characterized in that the bioavailability of metaxalone is independent of whether the composition is administered to the patient with food or on an empty stomach. The composition of the present invention is packaged in combination with written instructions, which instructions provide that the composition may be taken equally with or without food. Thus, the pharmaceutical composition as herein described, may be orally administered to humans on an empty stomach or with meals. The composition of the present invention is characterized in that it has an enhanced bioavailability as compared to conventional pharmaceutical composition of metaxalone available commercially. Bioavailability referred to herein is rate and extent to which the active drug ingredient, metaxalone, is absorbed into the systemic circulation from the pharmaceutical composition of the present invention.

The pharmaceutical compositions of the present invention may further include an analgesically effective amount of a non-steroidal anti-inflammatory drug, wherein said nonsteroidal anti-inflammatory drug comprises a propionic acid derivative, acetic acid derivative, fenamic acid derivative, biphenylcarboxylic acid derivative or an oxicam, or the pharmaceutically acceptable salts thereof. The propionic acid derivatives that may be used in the present invention include ibuprofen, naproxen, benoxaprofen, flurbiprofen, fenoprofen, ibuprofen aluminum, fenbufen, ketoprofen, pirprofen, carprofen, oxaprozin, pranoprofen, miroprofen, tioxaprofen, suprofen, alminoprofen, tiaprofenic acid, fluprofen or bucloxic acid. The acetic acid derivatives that may be used in the present invention include indomethacin, sulindac, tolmetin, diclofenac, fenclofenac, alclofenac, ibufenac, isoxepac, furofenac, tiopinac, zidometacin, acemetacin, fentiazac, clidanac or oxepinac. The fenamic acid derivatives that may be used in the present invention include mefenamic acid, meclofenamic acid, flufenamic acid, niflumic acid or tolfenamic acid. The biphenylcarboxylic acid derivatives that may be used in the present invention include diflunisal or flufenisal. The oxicams that may be used in the present invention include piroxicam, sudoxicam or isoxicam.

While the invention has been described with reference to specific embodiments, this was done for purposes of illustration only and should not be considered to limit the spirit or the scope of the invention.

### EXAMPLE 1

This example illustrates one embodiment of the pharmaceutical composition of the present invention. Tablets were prepared as per the formula given in Table 2 below.

TABLE 2

| | | Quantity (percent |
|---|---|---|
| | Quantity | by weight |
| Ingredients | (mg) | of the tablet) |
| Metaxalone (micronized) | 400.0 | 86.96 |
| Hydroxypropyl methyl cellulose E15LV | 4.00 | 0.87 |
| Pregelatinised starch (Starch 1500) | 30.00 | 6.52 |
| Iron oxide red | 0.3 | 0.065 |
| Sodium lauryl sulfate | 0.60 | 0.13 |
| Hydroxypropyl methyl cellulose E15LV | 2.50 | 0.54 |
| Colloidal Silicon dioxide | 0.75 | 0.163 |
| Corn starch (dried) | 16.35 | 3.55 |
| Magnesium stearate | 5.50 | 1.19 |

Metaxalone was micronized using a jet mill (MIDAS micronized M-200). Volume size distribution of the milled metaxalone as determined by Sympatec HELOS(H0899) particle size analyzer is given in Tables 3 and 4 below.

TABLE 3

| Cumulative | |
|---|---|
| % Undersize | Size (μm) |
| 36.67 | 1.80 |
| 45.83 | 2.20 |
| 53.91 | 2.60 |
| 60.94 | 3.00 |
| 69.92 | 3.60 |
| 79.39 | 4.40 |
| 86.29 | 5.20 |
| 92.06 | 6.20 |
| 96.07 | 7.40 |
| 98.12 | 8.60 |
| 99.23 | 10.00 |
| 99.80 | 12.00 |
| 100.00 | 15.00 |

TABLE 4

| Cumulative | |
|---|---|
| % Undersize | Size/diameter (μm) |
| 50% (d50) | 2.41 |
| 75% (d75) | 4.03 |
| 90% (d90) | 5.84 |
| 95% (d95) | 7.08 |
| 97% (d97) | 7.95 |
| 99% (d99) | 9.71 |

Tablets were prepared as per the following procedure. Hydroxypropyl methylcellulose E15LV, pregelatinised starch and iron oxide red were mixed together and passed through # 60 sieve (as defined by American Society for Testing and Materials, ASTM). Metaxalone (micronized) was mixed well with the above mix. Sodium lauryl sulfate was dissolved in distilled water and added to this blend while mixing. Hydroxypropyl methylcellulose E15LV dispersed in distilled water was used to granulate the powder blend. The granules thus obtained were dried in fluid bed dryer and passed through a mill. A mixture of starch, magnesium stearate and colloidal silicon dioxide, passed through a # 60 sieve, was then used to lubricate the dry granules. This lubricated mass was then compressed at a weight of 460 mg using 11.0 mm beveled edged round punches to obtain the final tablets.

The tablets thus obtained were tested for disintegration time as per standard procedure described in Indian Pharmacopoeia. It was observed that the tablet completely eroded into microparticles in the disintegration medium in about 30 minutes.

The tablets were subjected to dissolution testing using United States Pharmacopoeia type II dissolution apparatus at 75 rpm. The dissolution medium used was 900 ml of 1% sodium lauryl sulfate solution. The results of the dissolution test are mentioned in Table 5 below.

TABLE 5

| Time (minutes) | % drug released (±SD) |
|---|---|
| 15 | 23 ± 3.5 |
| 30 | 44 ± 6.44 |
| 60 | 71 ± 7.34 |
| 120 | 98 ± 2.58 |

It was observed that the tablet completely eroded in the dissolution medium in about 60 minutes into microparticles.

### EXAMPLE 2

The bioavailability of the pharmaceutical composition of the present invention (400 mg metaxalone tablets) and that of conventional pharmaceutical composition of metaxalone available commercially (Skelaxin®, 400 mg tablets) were studied. A single-dose, open label, randomized, comparative and two-way crossover pharmacokinetic study with a seven day washout period, was undertaken for the same.

Metaxalone (SPARC, Mumbai, Lot no. M274-3D, Mfg. Date: March 2001) 400 mg tablets was used as the test product and Skelaxin (Carnrick Lab Inc., USA, Lot no. GS 1043A, Exp. Date: October 2003) 400 mg tablets was used as the reference product.

Nine healthy male volunteers were enrolled for the study and all of them completed the two-way crossover study. The subjects were fasted overnight before dosing and for 4 hours thereafter. Drinking water was prohibited 2 hours before dosing and 2 hours thereafter. Standard meals were provided at 4 hours and 8 hours after dosing and at appropriate times thereafter. Meal plans were identical for both the periods.

Subjects received a single conventional tablet of metaxalone (400 mg) as the test product, and single conventional tablet of Skelaxin (400 mg) as the reference product with 240 ml of drinking water at ambient temperature after the overnight fast.

The pharmacokinetic assessment was based on the plasma levels of metaxalone measured by blood sampling. Blood samples were obtained before dosing and at the following times after administration of both the reference and test medications −0.5, 1, 1.5, 2, 2.5, 3, 3.5, 4, 4.5, 5, 6, 8, 12, 16, 24, 36 and 48 hours.

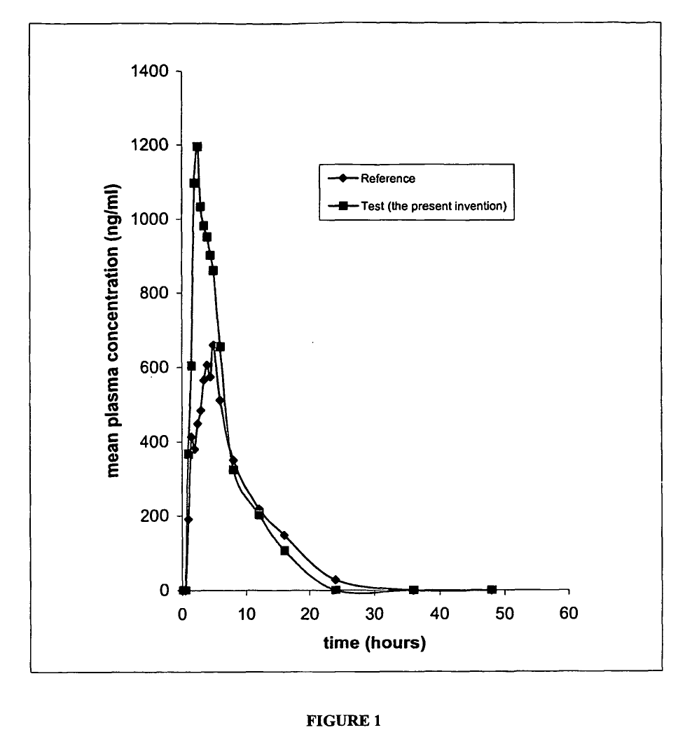

The plasma concentration of metaxalone was determined for samples collected at different time points and averaged over the nine volunteers. The data is given in Table 6 below. The plasma concentration versus time profile is illustrated in FIG. 1.

TABLE 6

| | Mean Plasma Concentrations (ng/ml) | |
|---|---|---|
| | Metaxalone 400 mg tablets | Skelaxin 400 mg tablets |
| Time (hrs) | (Test product) | (Reference product) |
| 0.0 | 0.00 | 0.00 |
| 0.5 | 0.00 | 0.00 |
| 1.0 | 367.63 | 191.26 |
| 1.5 | 604.73 | 413.47 |
| 2.0 | 1098.11 | 380.88 |
| 2.5 | 1196.08 | 449.43 |
| 3.0 | 1034.09 | 484.45 |
| 3.5 | 982.32 | 566.00 |
| 4.0 | 951.62 | 606.26 |
| 4.5 | 901.85 | 574.38 |
| 5.0 | 859.79 | 659.52 |
| 6.0 | 655.77 | 511.94 |
| 8.0 | 324.92 | 350.78 |
| 12.0 | 203.37 | 218.48 |
| 16.0 | 107.00 | 147.98 |
| 24.0 | 0.00 | 28.35 |
| 36.0 | 0.00 | 0.00 |
| 48.0 | 0.00 | 0.00 |

The pharmacokinetic parameters calculated using the Win Nonlin software are given in Tables 7 and 8 below.

TABLE 7
Untransformed

| | | Least Square Means | | |
|---|---|---|---|---|
| | | Skelaxin | Metaxalone | |
| | | Tablets | Tablets | |
| | | (Reference | (Test | |
| Parameter | Units | product) | product) | Ratio (% T/R) |
| Cₘₐₓ | ng/ml | 982.44 | 1418.56 | 144.39 |
| AUC₀₋ₜ | hr*ng/ml | 5594.83 | 7176.69 | 128.27 |
| AUC_{0-info} | hr*ng/ml | 6890.74 | 8507.94 | 123.47 |
| Tₘₐₓ | hr | 3.50 | 2.44 | 69.71 |

TABLE 8
Ln-transformed

| | | Least Square Means | | |
|---|---|---|---|---|
| | | Skelaxin | Metaxalone | |
| | | Tablets | Tablets | |
| | | (Reference | Test | |
| Parameter | Units | product) | product) | Ratio (% T/R) |
| Cₘₐₓ | ng/ml | 825.60 | 1355.21 | 164.15 |
| AUC₀₋ₜ | hr*ng/ml | 4518.61 | 6012.08 | 133.05 |
| AUC_{0-inf} | hr*ng/ml | 5719.94 | 7110.43 | 124.31 |
| Tₘₐₓ | hr | 3.07 | 2.31 | 75.24 |

As is evident from the table, the metaxalone composition of the present invention gave significantly higher peak plasma concentration, which was achieved more rapidly than with the reference product. The bioavailability, as measured by the area under the plasma concentration—time profile, was significantly higher for the pharmaceutical composition of the present invention as compared to the reference product.

1. A pharmaceutical composition comprising metaxalone and at least one pharmaceutically acceptable excipient, characterized in that the pharmaceutical composition has enhanced oral bioavailability as compared to the conventional pharmaceutical composition of metaxalone available commercially when administered without food to a patient who has fasted.

2. A pharmaceutical composition as claimed in claim 1, wherein the metaxalone is in a pharmaceutically acceptable solubility-improved form.

3. A pharmaceutical composition as claimed in claim 2, wherein the solubility-improved form is micronised metaxalone.

4. A pharmaceutical composition as claimed in claim 2, wherein the solubility-improved form is a salt form of metaxalone.

5. A pharmaceutical composition as claimed in claim 2, wherein the solubility-improved form is a crystalline form of metaxalone.

6. A pharmaceutical composition as claimed in claim 2, wherein the solubility-improved form is amorphous metaxalone.

7. A pharmaceutical composition as claimed in claim 1, wherein the composition comprises a mixture of metaxalone and a solubilizing agent.

8. A pharmaceutical composition as claimed in claim 1, wherein the metaxalone comprises the following particle size distribution characteristics: 99% undersize value between 10 and 40 μm in diameter, characterised in that the pharmaceutical composition has enhanced oral bioavailability.

9. A pharmaceutical composition as claimed in claim 1, wherein the metaxalone has specific surface area per unit volume of more than 1.5 m²/cm³.

10. A pharmaceutical composition as claimed in claim 9, wherein the metaxalone has specific surface area per unit volume of more than 2.5 m²/cm³.

11. A pharmaceutical composition as claimed in claim 10, wherein the metaxalone has specific surface area per unit volume of more than 3.0 m²/cm³.

12. A pharmaceutical composition as claimed in claim 1, wherein the metaxalone comprises the following particle size distribution characteristics: 99% undersize value of 40 μm, 90% undersize value of 30 μm, and 50% undersize value of 10 μm.

13. A pharmaceutical composition as claimed in claim 1, wherein the metaxalone comprises the following particle size distribution characteristics: 99% undersize value of 30 μm, 90% undersize value of 14 μm, and 50% undersize value of 6 μm.

14. A pharmaceutical composition as claimed in claim 1, wherein the metaxalone comprises the following particle size distribution characteristics: 99% undersize value of 10 μm, 90% undersize value of 5 μm, and 50% undersize value of 3 μm.

15. A pharmaceutical composition as claimed in claim 1, wherein the amount of metaxalone is in the range of 400 mg to 1600 mg.

16. A pharmaceutical composition as claimed in claim 1, wherein the pharmaceutically acceptable excipient comprises a wetting agent.

17. A pharmaceutical composition as claimed in claim 16, wherein the wetting agent comprises a surfactant.

18. A pharmaceutical composition as claimed in claim 17, wherein the surfactant comprises sodium lauryl sulfate.

19-22. (canceled)

23. A pharmaceutical composition as claimed in claim 1 wherein the pharmaceutical composition further comprises an analgesically effective amount of a non-steroidal anti-inflammatory drug, wherein said nonsteroidal anti-inflammatory drug comprises a propionic acid derivative, acetic acid derivative, fenamic acid derivative, biphenylcarboxylic acid derivative or an oxicam, or the pharmaceutically acceptable salts thereof.

24. A pharmaceutical composition as claimed in claim 2 wherein the pharmaceutical composition further comprises an analgesically effective amount of a non-steroidal anti-inflammatory drug, wherein said nonsteroidal anti-inflammatory drug comprises a propionic acid derivative, acetic acid derivative, fenamic acid derivative, biphenylcarboxylic acid derivative or an oxicam, or the pharmaceutically acceptable salts thereof.

25-26. (canceled)

27. A pharmaceutical composition comprising metaxalone and at least one pharmaceutically acceptable excipient, wherein at least 99% of the metaxalone has a particle size not more than about 10 μm in diameter.

28. A pharmaceutical composition in solid dosage form comprising an effective amount of metaxalone and at least one pharmaceutically acceptable excipient wherein the dosage form has an enhanced oral bioavailability as compared to the metaxalone product of New Drug Application No. 13-217.
